(19) United States
(12) Patent Application Publication
(10) Pub. No.: US 2025/0229034 A1
(43) Pub. Date: Jul. 17, 2025

(54) Automatic feedback mechanism for a medicament delivery device

(71) Applicant: SHL Medical AG, Zug (CH)

(72) Inventors: Christopher Foster, Zug (CH); Daniel Carlsson, Nacka Strand (SE); Daniel Säll, Nacka Strand (SE)

(21) Appl. No.: 18/700,460

(22) PCT Filed: Oct. 6, 2022
(86) PCT No.: PCT/EP2022/077868

§ 371 Date: Apr. 11, 2024

(30) Foreign Application Priority Data
Oct. 19, 2021 (EP) 21203578.6

(51) Int. Cl. A61M 5/315; A61M 5/20

(52) U.S. Cl. CPC A61M 5/3157; A61M 5/2033; A61M 2205/581; A61M 2205/582

(57) ABSTRACT

The present disclosure provides a feedback mechanism for a medicament delivery device, the feedback mechanism includes (i) a housing structure including a holding arm, (ii) a plunger rod configured to engage with the holding arm in a pre-tensioned state, and (iii) a signal generating member attached to the housing structure, the signal generating member including a resilient tongue which in the pre-tensioned state of the plunger rod is arranged in contact with a first portion of the plunger rod. The plunger rod includes a second portion arranged distally from the first portion, the second portion including a cut-out. The resilient tongue is operably arranged to, upon movement of the plunger rod in the proximal direction, be brought into contact with the second portion of the plunger rod to interact with the cut-out for generating an audible and/or tactile feedback.

### TECHNICAL FIELD

The present disclosure generally relates to a medicament delivery device for expelling medicament from a flexible medicament container, and in particular to an automatic feedback mechanism for a medicament delivery device.

### BACKGROUND

A number of medical conditions require reliable provision of a medicament from a medicament delivery device. The medicament is typically comprised in a medicament container within the medicament delivery device, the medicament container being configured to expel the medicament via some type of delivery member, such as a needle or a nozzle.

These days, a number of different medicament delivery devices exist, including various types of injectors (for example pen injectors, autoinjectors, on-body devices). Although many of these devices have enabled major improvements in the management of a number of medical conditions, various drawbacks do still exist in the current technology.

When handling a medicament delivery device, it may be beneficial to provide an indication to a user regarding the current state of the medicament delivery device. For example, it is typically required that an automated medicament delivery device is maintained in position at the injection site until the expelled dose has been properly absorbed and the liquid pressure created by the injected dose has subsided in the injection site area. To this end, for automated medicament delivery devices it may be advantageous for a user to know when the dose expulsion commences, facilitating determination of when the medicament delivery device may be removed from the injection site. US2016/0008542 discloses an auto-injector for dispensing a liquid product with the intention to generate an audible and/or tactile signal. The device includes a housing, a container, a blocking sleeve, a drive member, a spring, a needle protection sleeve, and a holding element supporting the spring. In the initial state, the holding element is engaged with the drive member, which holding element is further maintained in position by the inner circumference of the blocking sleeve. To administer the product from the container the needle protection sleeve is moved by an activation stroke. The holding element is thereby disengaged from the drive member, and is instead set into engagement with the blocking sleeve. The drive member is thereby released. The holding element can hence be moved some distance relative to the housing, whereby the holding element transports the blocking sleeve. This causes the blocking sleeve to strike against a start signal stop formed by a mechanical holder. An audible and/or tactile signal is thereby emitted and signals the user of the device that dispensing of the product has started.

The disclosure of US2016/0008542 provides a solution for a specific configuration or design of an auto-injector. Moreover, the audible and/or tactile signal is not really provided at the time when liquid expulsion commences.

### SUMMARY

An object of the present disclosure is thus to provide an automatic feedback mechanism for a medicament delivery device which solves, or at least mitigates, problems of the prior art.

There is hence according to a first aspect of the present disclosure provided an automatic feedback mechanism for a medicament delivery device, the automatic feedback mechanism comprising: a housing structure having a proximal end and a distal end, the housing structure comprising a holding arm arranged at the distal end of the housing structure; a plunger rod having a proximal end and a distal end, the plunger rod comprising a holding structure arranged at the distal end of the plunger rod, the holding structure being configured to engage with the holding arm in a pre-tensioned state, wherein the plunger rod is operatively arranged to, upon activation, be released from its pre-tensioned state by disengagement of the holding structure from the holding arm of the housing structure, to move proximally inside the housing structure; a signal generating member attached to the housing structure, the signal generating member being arranged radially outside of the plunger rod and comprising a resilient tongue which in the pre-tensioned state of the plunger rod is arranged in contact with a first portion of the plunger rod; wherein the plunger rod comprises a second portion arranged distally from the first portion, the second portion comprising a cut-out, and wherein the resilient tongue is operably arranged to, upon movement of the plunger rod in the proximal direction, be brought into contact with the second portion of the plunger rod to interact with the cut-out for generating an audible and/or tactile feedback.

An effect obtainable thereby is that improved automatic feedback mechanism may be provided. Thus, the automatic feedback mechanism notifies a user of the medicament delivery device of expulsion of medicament from a medicament container. The audible and/or tactile feedback may be referred to as an audible and/or tactile signal.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to one embodiment, the plunger rod is, upon activation, movable between a first end position and a second end position, wherein the resilient tongue is arranged in pre-tensioned contact with the first portion of the plunger rod in the first end position of the plunger rod, and is arranged to interact with the cut-out for generating an audible and/or tactile feedback in the second end position of the plunger rod. Thus, the automatic feedback mechanism may notify a user of the medicament delivery device of and end of expulsion of medicament from a medicament container.

According to one embodiment, the second portion of the plunger rod is arranged in a distal portion of the plunger rod. According to one embodiment, the first portion of the plunger rod is arranged in a proximal portion of the plunger rod.

According to one embodiment, the first portion of the plunger rod comprises a cut-out which the resilient tongue is in pre-tensioned contact with in the pre-tensioned state of the plunger rod. Hereby, upon movement of the plunger rod in the proximal direction, the resilient tongue will be forced out from the cut-out of the first portion of the plunger rod, typically by tensioning of the resilient tongue. Such movement, or tensioning, of the resilient tongue may result in a tactile feedback and thereby notify a user of the medicament delivery device of a start of expulsion of medicament from a medicament container.

According to one embodiment, the plunger rod comprises an external surface having an intermediate portion arranged axially between the first portion and the second portion, wherein the intermediate portion is arranged to support and flex the resilient tongue radially outwards upon movement of the plunger rod in the proximal direction. Thus, prior to reaching the cut-out of second portion of the plunger rod, the resilient tongue is forced radially outwards by the intermediate portion of the plunger rod, thereby tensioning the resilient tongue, said tensioning being at least partly released as the resilient tongue interacts with the cut-out. Hereby, an improved audible and/or tactile feedback is provided. Stated differently, the plunger rod is operably arranged to, upon activation, move axially in the proximal direction such that the resilient tongue flexes outwards and slides along the intermediate portion prior to reaching and interacting with the cut-out of the second portion.

According to one embodiment, the diameter of the plunger rod at the intermediate portion is larger than the diameter of the plunger rod at the cut out of the second portion. Thus, a simple yet effective structure for tensioning the resilient tongue prior to its interaction with the cut-out of the second portion, as well as an efficient release of the built-up tension upon interaction with the cut-out of the second portion, are provided.

According to one embodiment, the cut-out of the second portion of the plunger rod is an indentation in the plunger rod. Hereby, a simple way of providing different cross-sections, or different diameters, of different portions of the plunger rod is provided. Thus, the resilient tongue may be tensioned and released in an advantageous manner.

According to one embodiment, the resilient tongue is arranged to, upon movement of the plunger rod in the proximal direction, be brought into contact with the indentation for generating the audible and/or tactile feedback. Thus, the resilient tongue may interact with the indentation for generating the audible and/or tactile feedback upon being brought into contact with the second portion of the plunger rod. The indentation typically comprises a bottom surface which the resilient tongue encounters as it flexes inwards into the cut-out. The encountering with the bottom surface may thus cause the audible and/or tactile feedback.

According to one embodiment, the cut-out of the second portion of the plunger rod is a through hole in the plunger rod. A through hole enables that a larger force of the built-up tension in the resilient tongue can be released compared to if the cut-out is an indentation, as the resilient tongue may reach further radially inwards into, and radially beyond, the plunger rod. Thus, the resilient tongue may be brought into contact with an interior element arranged radially inwards of the plunger rod.

According to one embodiment, the feedback mechanism further comprises a drive spring operably arranged to act on the plunger rod to exert a force in the proximal direction, wherein the plunger rod is arranged radially outwards of the drive spring. Such configuration of the plunger rod and the drive spring is advantageous for generating the audible and/or tactile feedback previously described. Thus, as the plunger rod is arranged radially outwards of the drive spring, the signal generating member may be arranged adjacent the plunger rod, such as e.g. radially outwards of the plunger rod. Hereby, a less complex configuration of the signal generating member and the plunger rod is provided. The plunger rod may have a cylindrical body, or be tubularly shaped. Stated differently, the plunger rod may be formed as a hollow cylinder, wherein the drive spring is arranged inside the hollow cylinder.

According to one embodiment, the resilient tongue is arranged to, upon movement of the plunger rod in the proximal direction, be brought into contact with the drive spring via the through hole for generating the audible and/or tactile feedback. Thus, the resilient tongue may interact with the drive spring arranged radially inwards of the plunger rod for generating the audible and/or tactile feedback upon being brought into contact with the second portion of the plunger rod. The through hole enable the resilient tongue to reach the drive spring. The encountering with the drive spring thus causes the audible and/or tactile feedback.

According to one embodiment, the signal generating member comprises a sleeve surrounding the plunger rod, wherein the resilient tongue protrudes radially inwards from the sleeve. Hereby, the resilient tongue may be tensioned by being forced radially outwards towards the sleeve, and may be released from its tensioned state by being allowed to flex radially inwards from the sleeve. Moreover, the sleeve may support the resilient tongue to ensure a reliable function thereof.

According to one embodiment, the resilient tongue is a first resilient tongue, and the signal generating member comprises a second a resilient tongue protruding radially inwards from the sleeve opposite to the first resilient tongue. Hereby, the audible and/or tactile feedback may be further improved. Typically, the cut-out in the second portion of the plunger rod is a first cut-out in the second portion of the plunger rod, wherein the plunger rod further comprises a second cut-out in the second portion. Thus, the first cut-out of the second portion is arranged to interact with the first resilient tongue as previously described, and the second cut-out of the second portion is arranged to interact with the second resilient tongue. Typically, the first and second cut-outs of the second portion are arranged on opposite side of the plunger rod.

According to one embodiment, the resilient tongue is formed as a coil within the housing structure. A coil may be tensioned in a reliable manner by expanding, and may be released from its tensioned state in a reliable manner by partly contracting. As previously described, upon movement of the plunger rod in the proximal direction, the coil may be expanded as it radially aligns with the intermediate portion of the plunger rod, and may contract to generate the audible and/or tactile feedback as it encounters the cut-out of the second portion of the plunger rod.

According to one embodiment, the signal generator member comprises an attachment arm extending distally from the sleeve, wherein the attachment arm is fixedly attached to the housing structure. Thus, the sleeve may be fixedly attached to the housing structure by means of said attachment arm in order to ensure a fixed axial position of the signal generating member within the housing structure.

According to one embodiment, the holding arm of the housing structure is a first holding arm, wherein the housing structure further comprises a second holding arm arranged at the distal end of the housing structure. Thus, the holding structure of the plunger rod is configured to engage with the first and second holding arms in a pre-tensioned state, wherein the plunger rod is operatively arranged to, upon activation, be released from its pre-tensioned state by disengagement of the holding structure from the first and second holding arms of the housing structure, to move proximally inside the housing structure.

There is according to a second aspect of the present disclosure provided a medicament delivery device comprising a feedback mechanism according to the first aspect of the present disclosure.

Effects and features of the second aspect of the invention are largely analogous to those described above in connection with the first aspect of the invention. Embodiments mentioned in relation to the first aspect of the invention are largely compatible with the second aspect of the invention.

The medicament delivery device may furthermore comprise an actuator having at least one actuator arm, or release arm, configured to interact with the holding arm or holding arms. For example, the at least one release arm is configured to interact with the holding arm(s) to radially force the holding arm(s) outwards, whereby the holding arm(s) is/are disengaged from the holding structure of the plunger rod causing the plunger rod to be released from its pre-tensioned state. Thus, the at least one release arm is configured to displace the holding arm(s) from the holding structure.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 schematically shows a perspective view of an example of a medicament delivery device;

FIG. 2 shows an exploded view of the medicament delivery device in FIG. 1;

FIGS. 3A and 3B show schematic cross sectional views of a distal portion of the medicament delivery device in FIG. 2;

FIG. 4 is a schematic cross sectional view of an example automatic feedback mechanism for a medicament delivery device;

FIG. 5A is a perspective part cross-sectional view of a signal generating member of the automatic feedback mechanism of FIG. 4;

FIG. 5B is a perspective view of a plunger rod of the automatic feedback mechanism of FIG. 4;

FIGS. 6A-6C are cross-sectional views of the automatic feedback mechanism of FIG. 4 describing the operation of the automatic feedback mechanism;

FIG. 7A is a perspective view of yet an example plunger rod of the automatic feedback mechanism;

FIG. 7B is a cross-sectional view of the plunger rod of FIG. 7A; and

FIGS. 8A and 8B are perspective part cross-sectional views of an example embodiment of a part of the medicament delivery device of FIG. 2.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

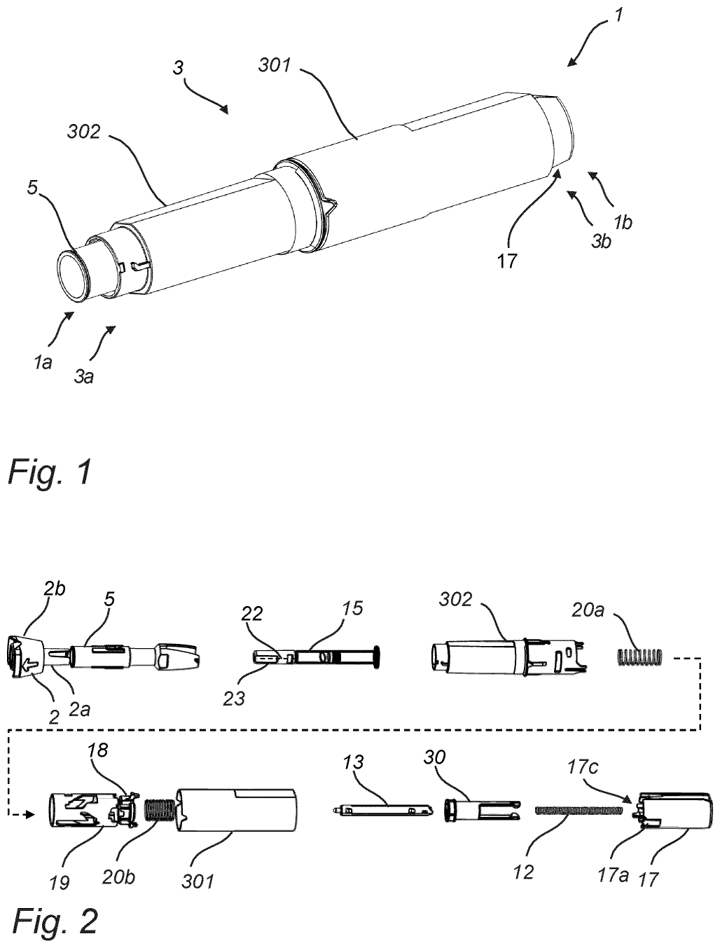

FIG. 1 shows an example of a medicament delivery device 1 configured to expel medicament from a medicament container structure. The medicament delivery device 1 comprises a proximal end 1a and a distal end 1b.

The medicament delivery device 1 comprises a housing structure 3. The housing structure 3 has a proximal end 3a and a distal end 3b. In the embodiment of FIG. 1, the housing structure 3 is divided into an upper housing 301 and a lower housing 302.

The medicament delivery device 1 comprises a delivery member cover 6. The delivery member cover 6 is arranged in the housing structure 3 and extends proximally from the proximal end 3a of the housing structure 3.

The delivery member cover 6 is configured to be moved linearly relative to the housing structure 3 from a protracted position shown in FIG. 1 to a retracted position in which the delivery member cover 6 is received further in the housing structure 3. The delivery member cover 6 is biased in the proximal direction towards the protracted position.

The medicament delivery device 1 is brought into an activation admittable state in which discharge of medicament from the medicament container is enabled by movement of the delivery member cover 6 further into the housing structure 3 towards the retracted position. The activation of discharge of medicament from the medicament container is achieved by an actuator 17, described more in detail later in the text.

FIG. 2 shows an exploded view of the medicament delivery device 1. The medicament delivery device is configured to expel medicament from a medicament container 15 via a delivery member 22 (shown as a dashed line). In FIG. 2, the delivery member 22 is protected by a ring needle shield, RNS, 23. The delivery member 22 may e.g. be a needle. First, the general configuration of the medicament delivery device 1 will be described, then embodiments of an automatic feedback mechanism of the medicament delivery device 1 will be described in greater detail. The automatic feedback mechanism of the medicament delivery device 1 may be defined by combining specific components of the medicament delivery device 1.

From left to right, the medicament delivery device 1 comprises the following components.

The medicament delivery device 1 comprises a cap 2 removably arranged relative the delivery member cover 6. In the embodiment of FIG. 2, the cap 2 comprises a gripping portion 2b and a protrusion 2a extending in the longitudinal direction distally of the gripping portion 2b. The gripping portion 2b comprises a gripping surface which a user may interact with in order to remove the cap 2 from the delivery member cover 6. The protrusion 2a is arranged to extend into the delivery member cover 6 to interact with the RNS 23 of the medicament container 15.

The medicament delivery device 1 comprises the lower housing 302 as shown in FIG. 1. The lower housing 302 is configured to primarily house the delivery member cover 6 and the medicament container 15.

The medicament delivery device 1 comprises a first resilient member 20a in the form of a first coil spring 20a, a rotator 19, a collar 18, and a second resilient member 20b in the form of a second coil spring 20b. The first resilient member 20a is arranged to exert a biasing force in the proximal direction on the delivery member cover 6.

The medicament delivery device 1 comprises the upper housing 301 shown in FIG. 1. The upper housing 301 is configured to house at least a distal end of the lower housing 302, the rotator 19 and the collar 18.

The medicament delivery device 1 comprises a plunger rod 13 operably arranged to, upon activation, move in the proximal direction. Thus, in the embodiment of FIG. 2, the plunger rod 13 is configured to be moved axially inside the housing structure 3. During a medicament delivery operation, the plunger rod 13 moves from a first axial position towards the medicament container 15 for expelling the medicament. The plunger rod 13 may have a cylindrical body.

The medicament delivery device 1 comprises a signal generating member 30, arranged to at least partly guide movement of the plunger rod 13 in the axial direction.

The medicament delivery device 1 comprises a drive spring 12 configured to bias the plunger rod 13 in the proximal direction. The plunger rod 13 is axially fixed relative to the housing structure 3 in the first axial position prior to a medicament delivery operation.

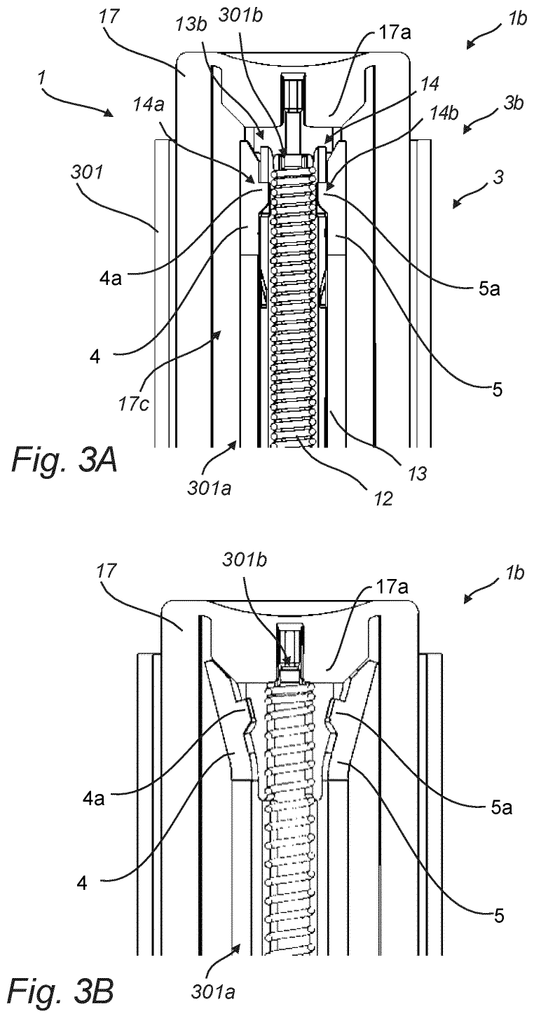

The medicament delivery device 1 comprises the actuator 17 described with reference to FIG. 1. The actuator 17 comprises a receiving recess 17c arranged to receive at least the drive spring 12 and the plunger rod 13. Moreover, the actuator 17 comprises at least one actuator arm, or release arm 17a, which is better shown in FIGS. 3A and 3B, and further described below.

The second resilient member 20b is compressed between the collar 18 and the actuator 17. The second resilient member 20b serves at least two purposes. One purpose is to bias the collar 18 in the proximal direction towards the medicament container 15 to achieve a dampening effect of the medicament container 15. The dampening effect reduces the risk of damaging the medicament delivery device 1 if it e.g. is dropped to the floor. According to one embodiment, a separate dampener is arranged in between the collar 18 and the second resilient member 20b. The second purpose of the second resilient member 20b is to bias the actuator 17 in the distal direction. Hereby, rattling of the actuator 17 is reduced, and a force pushing the actuator 17 to its most distal position is provided. The second resilient member 20b is preferably a weaker coil spring compared to the first resilient member 20a.

FIGS. 3A and 3B are schematic cross-sectional views of a distal portion (i.e. a portion at the distal end 1b) of the medicament delivery device 1 of FIGS. 1 and 2, why the same reference numerals are used.

The housing structure 3 comprises at least one holding arm 4, 5 arranged at the distal end 3b of the housing structure 3. In the present embodiment, two holding arms 4, 5 are arranged at the distal end 3b of the housing structure 3. In more detail, the upper housing 301 of the housing structure 3 comprises an inner sleeve portion 301a from which the two holding arms 4, 5 extends in the distal direction. The inner sleeve portion 301a and the two holding arms 4,5 are arranged radially inwards of the actuator 17 inside the receiving recess 17c. Each one of the two holding arms 4, 5 comprises a holding protrusion 4a, 5a. The plunger rod 13 comprises a holding structure 14 arranged at the distal end 13b of the plunger rod 13, the holding structure 14 being configured to engage with the two holding arms 4, 5 to hold the plunger rod 13 in a pre-tensioned state. The plunger rod 13 is pre-tensioned in the proximal direction by means of the drive spring 12. The housing structure 3 further comprises a bridge 301b forming a part of the upper housing 301. The bridge 301b is arranged to support a distal end of the drive spring 12. The bridge 301b is described in detail further below and with reference to FIG. 9.

In more detail, the holding structure in the embodiment of FIGS. 3A and 3B comprises two radial openings 14a, 14b arranged on opposite lateral sides of the plunger rod 13. The two holding protrusions 4a, 5a extends radially inwards into the two radial openings 14a, 14b to lock the plunger rod 13 in its pre-tensioned state, as shown in FIG. 3A. The holding arms 4, 5 are operatively arranged to, upon activation by the at least one release arm 17a, flex radially outwards such that the holding protrusions 4a, 5a are withdrawn from the radial openings 14a, 14b to release the plunger rod 13, as shown in FIG. 3B. According to one embodiment, in order for the at least one arm 17a of the actuator 17 to be allowed to move in the proximal direction to release the plunger rod 13, the rotator 19 is first arranged in an activation admittable state by the delivery member cover 6. That is, as the delivery member cover 6 is brought into its retracted position, a distal end 5a of the delivery member cover 6 may engage the rotator 19 and thereby transmits the axial movement of the delivery member cover 6 into a rotational movement of the rotator 19. Hereby, the rotator 19 is rotated into its activation admittable state, enabling the at least one arm 17a of the actuator 17 to be allowed to move in the proximal direction.

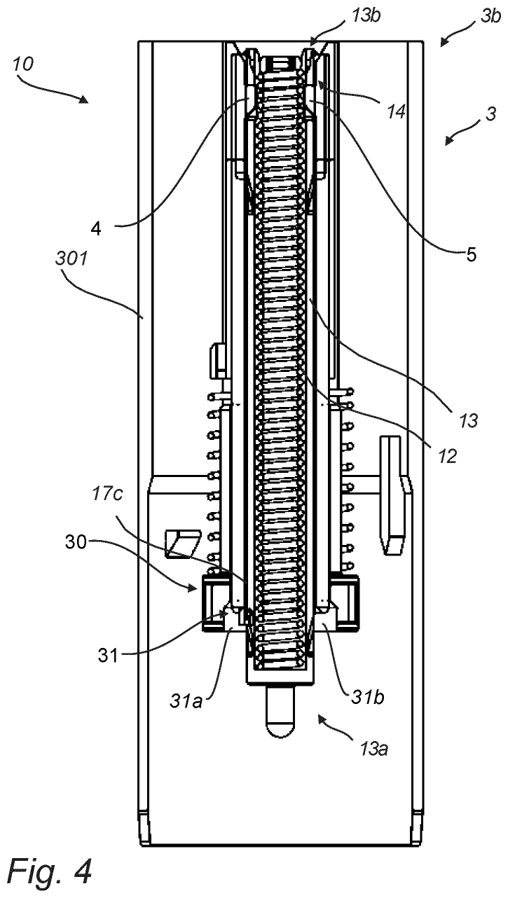

FIG. 4 is a schematic cross sectional view of an automatic feedback mechanism 10 for the medicament delivery device 1 of FIGS. 1 and 2, why the same reference numerals as for the medicament delivery device 1 are used. The automatic feedback mechanism 10 comprises the housing structure 3 having a proximal end 3a and a distal end 3b (of which only the proximal end 3b is shown in FIG. 4). The housing structure 3 shown in FIG. 4 is the upper housing 301. Thus, according to one embodiment, the automatic feedback mechanism 10 comprises the upper housing 301. The housing structure 3 comprises the two holding arms 4, 5 described with reference to FIGS. 3A-3B.

The automatic feedback mechanism 10 comprises the plunger rod 13 having a proximal end 13a and a distal end 13b. Furthermore, the plunger rod 13 comprises the holding structure 14 arranged at the distal end 13b of the plunger rod 13, the holding structure 14 being configured to engage with the two holding arms 4, 5 in a pre-tensioned state of the plunger rod 13 as described with reference to FIGS. 3A and 3B. The drive spring 12 is configured to bias the plunger rod 13 in the proximal direction. The plunger rod 13 is operatively arranged to, upon activation, be released from its pre-tensioned state by disengagement of the holding protrusion 14 from the two holding arms 4, 5, to move proximally inside the housing structure 3. The release of the plunger rod 13 is e.g. achieved by that the at least one release arm 17a of the actuator 17 engages the two holding arms 4, 5 and pushing them radially outwards.

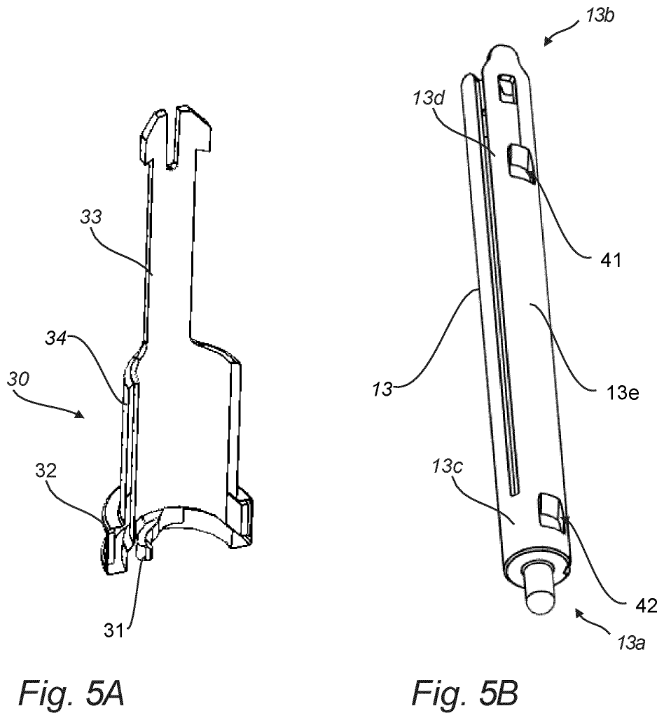

The automatic feedback mechanism 10 comprises the signal generating member 30 attached to the housing structure 3. The signal generating member 30 is arranged radially outside of the plunger rod 13 and comprises a resilient tongue 31 (better shown in FIGS. 5A-5B), which in the pre-tensioned state of the plunger rod 13 shown in FIG. 4, is arranged in contact with a first portion 13c of the plunger rod 13. In more detail, the resilient tongue 31 may be a first resilient tongue 31a, and the signal generating member 30 may comprise a second resilient tongue 31b, further described below.

The plunger rod 13 and the signal generating member 30 of the automatic feedback mechanism 10 will now be described with reference to FIGS. 5A and 5B.

FIG. 5A is a perspective part cross-sectional view of the signal generating member 30. The signal generating member 30 comprises a sleeve 32 arranged to surround or encompass the plunger rod 13. As FIG. 5A is showing a cross-sectional cut along the axial direction of the signal generating member 30, only half the circumference of the sleeve 32 is shown. The resilient tongue 31 protrudes radially inwards from the sleeve 32. Hereby, the resilient tongue 31 may interact with the plunger rod 13. The resilient tongue 31 is here formed as a coil, or part of a coil, as the resilient tongue extends at in the circumferential direction along the inner surface of the sleeve 32.

The signal generating member 30 further comprises an attachment arm 33 extending distally of the sleeve 32 for attachment to the housing structure 3. Thus, the sleeve 32 may be fixedly attached to the housing structure 3 by means of the attachment arm in order to ensure a fixed axial position of the signal generating member 30 within the housing structure 3. The signal generating member 30 may further comprise a mid portion 34 arranged in between the sleeve 32 and the attachment arm 33, and connecting the sleeve 32 with the attachment arm 33.

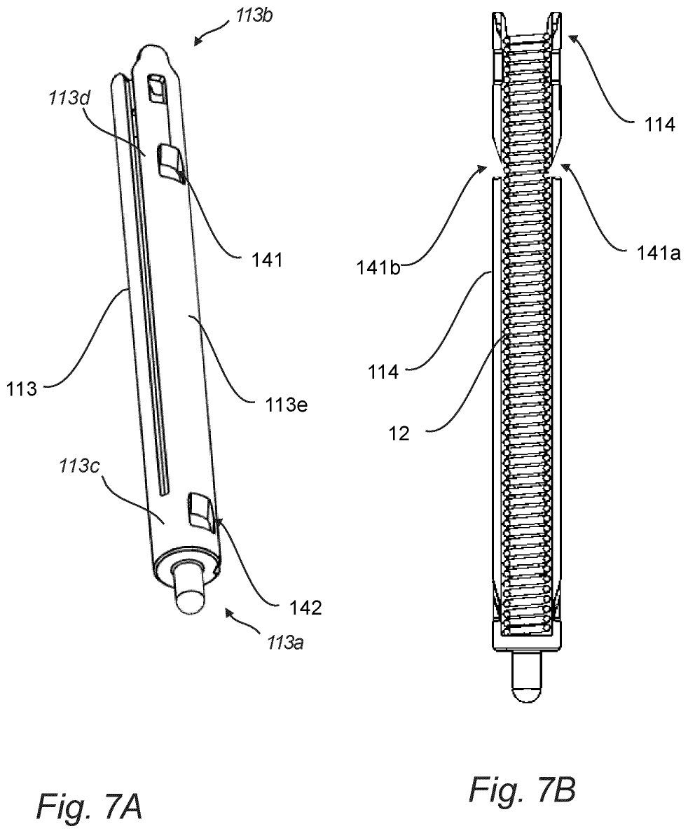

FIG. 5B is a perspective view of the plunger rod 13. The plunger rod 13 comprises a first portion 13c towards the proximal end 13a of the plunger rod 13, and comprises a second portion 13d towards the distal end 13b of the plunger rod 13. The first portion 13c comprises a cut-out 42, and the second portion 13d comprises a cut-out 41, wherein the resilient tongue 31 is arranged to interact with the cut-outs 41, 42 of the first and second portions 13c, 13d as will be described with reference to FIGS. 7A and 7B. Moreover, the plunger rod 13 comprises an external surface having an intermediate portion 13e arranged axially between the first portion 13c and the second portion 13d of the plunger rod 13. As seen in FIG. 5B, the cut-out 41 of the second portion 13d of the plunger rod 13 is an indentation. Correspondingly, the cut-out 42 of the first portion 13c of the plunger rod 13 is an indentation. Thus, the diameter of the plunger rod 13 at the intermediate portion 13e is larger than the diameter of the plunger rod 13 at the cut-out 41 of the second portion 13d, and the diameter of the plunger rod 13 at the cut-out 42 of the first portion 13c.

The plunger rod 13 may be symmetrical along a longitudinal cross-sectional cut. Thus, the cut-out 41 of the second portion 13d may be a first cut-out 41 of the second portion 13d, and a corresponding second cut-out of the second portion 13d may be arranged radially opposite the first cut-out 41. Similarly, the cut-out 42 of the first portion 13c may be a first cut-out 42 of the first portion 13c, and a corresponding second cut-out of the first portion 13c may be arranged radially opposite the first cut-out 42. Moreover, the resilient tongue 31 may be a first resilient tongue 31a, and the signal generating member 30 may comprises a second a resilient tongue 31b (as shown in FIG. 4) protruding radially inwards from the sleeve 32 opposite to the first resilient tongue 31a. Thus, the first resilient tongue 31a is arranged to interact with the first cut-outs 41, 42 of the first and second portions 13c, 13d, and the second resilient tongue 31b is arranged to interact with the second cut-outs of the first and second portions 13c, 13d. However, for increase intelligibility, only one the first resilient tongue 31, 31a and the first cut-outs 41, 42 of the first and second portions 13c, 13d are described in the following.

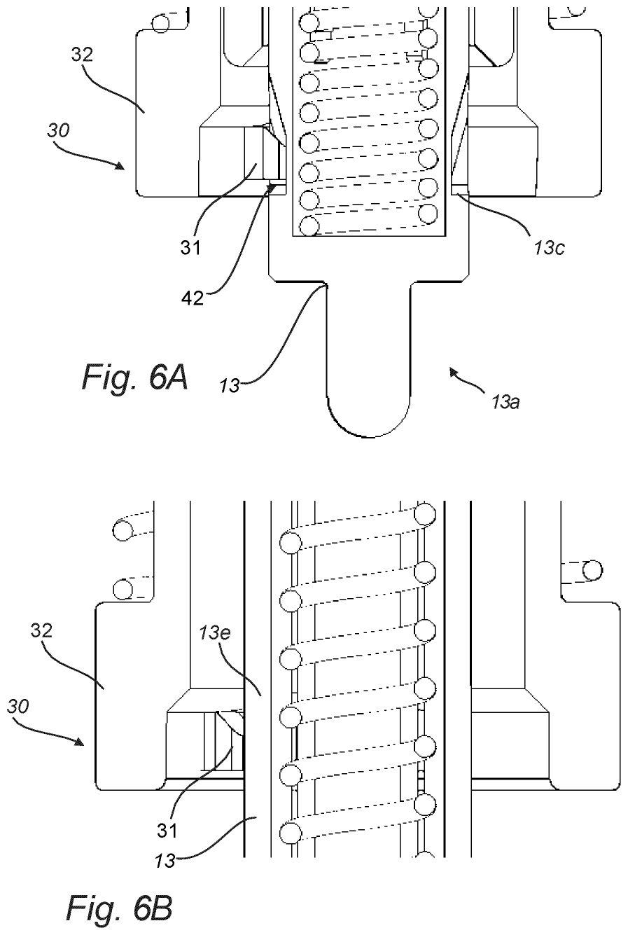

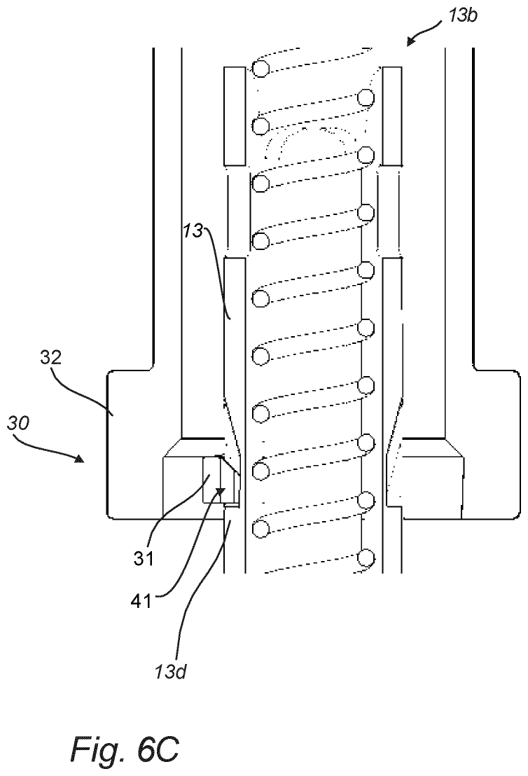

The operation of the automatic feedback mechanism 10 will now be described in further detail with reference to FIGS. 6A, 6B and 6C, together with the previously described FIG. 4 and FIGS. 5A and 5B.

FIG. 6A show a part of the automatic feedback mechanism 10 of FIG. 4. Here, the plunger rod 13 is arranged in its pre-tensioned state, as described with reference to FIG. 3A. Thus, the plunger rod 13 is arranged in a first end position. In FIG. 6A, the resilient tongue 31 is in a pre-tensioned contact with the cut-out 42 of the first portion 13c of the plunger rod 13.

The resilient tongue 31 is operably arranged to, upon movement of the plunger rod 13 in the proximal direction, be brought into contact with other portions of the plunger rod 13. Thus, as the plunger rod 13 moves in the proximal direction, the resilient tongue 31 will be forced to flex radially outwards as it leaves the cut-out 42 of the first portion 13c of the plunger rod 13.

FIG. 6B show the same automatic feedback mechanism 10 of FIG. 6A, but here the plunger rod 13 has moved further in the proximal direction. Thus, the resilient tongue 31 is brought into contact with the intermediate portion 13e. Stated differently, the intermediate portions 13e supports and flex the resilient tongue 31 radially outwards upon movement of the plunger rod 13 in the proximal direction.

In FIG. 6C, the same automatic feedback mechanism 10 of FIGS. 6A and 6B is shown, but here the plunger rod 13 has moved in the proximal direction to its second end position. Thus, in FIG. 6C, the medicament delivery operation is completed. The resilient tongue 31 interacts with the cut-out 41 of the second portion 13d of the plunger rod 13 for generating an audible and/or tactile feedback. That is, the plunger rod 13 has moved, upon activation, axially in the proximal direction from the first end position shown in FIG. 6A, further a state in which the resilient tongue 31 slides along the intermediate portion 13e, to finally reach the second end position for interaction with the cut-out 41 of the second portion 13d for generating the audible and/or tactile feedback. The cut-out 41 of the second portion is in FIG. 6C an indentation. Thus, the resilient tongue 31 is brought into contact with the indentation, and typically a bottom surface thereof, for generating the audible and/or tactile feedback.

As the diameter of the plunger rod 13 at the intermediate portion 13e is larger than the diameter of the plunger rod 13 at the cut-out 41 of the second portion 13d, and larger than the diameter of the plunger rod 13 at the cut-out 42 of the first portion 13c, the resilient tongue 31 is forced radially outwards by the intermediate portion 13e of the plunger rod 13, thereby tensioning the resilient tongue 31 upon movement of the plunger rod 13 in the proximal direction. The tensioning of the resilient tongue 31 is at least partly released as the resilient tongue 31 interacts with the cut-out 41 of the second portion 13d of the plunger rod 13 for generating the audible and/or tactile feedback.

FIG. 7A is a perspective view of an alternative plunger rod 113. The plunger rod 113 may interact with the signal generating member 30 of FIG. 5A in a corresponding manner as the plunger rod 13. The plunger rod 113 comprises a first portion 113c towards the proximal end 113a of the plunger rod 113, and comprises a second portion 113d towards the distal end 113b of the plunger rod 113, wherein an intermediate portion 113e is arranged therebetween. The plunger rod 113 comprises a holding structure 114 corresponding to the holding structure 14 of the plunger rod 13 of FIGS. 3A and 3B, not described here again. The first portion 113c comprises a cut-out 142, and the second portion 113d comprises a cut-out 141, wherein the resilient tongue 31 of the signal generating member 30 is arranged to interact with the cut-outs 141, 142 of the first and second portions 113c, 113d in a corresponding manner as described with reference to FIGS. 6A-6C. However, the cut-out 141 of the second portion 113d of the plunger rod 113 is a through-hole.

The plunger rod 113 of FIG. 7A may be symmetrical along a longitudinal cross-sectional cut in a corresponding manner as the plunger rod 13 of FIG. 5B. This is shown in FIG. 7B in which a first cut-out 141a and a second cut-out 141b is provided on opposite lateral sides of the plunger rod 113. Moreover, in FIG. 7B, the drive spring 12 of the medicament delivery device 1 is shown. Thus, the drive spring 12 is operably arranged to act on the plunger rod 113 to exert a force in the proximal direction. The drive spring 12 is arranged radially inwards of the plunger rod 113. Thus, in the second end position of the plunger rod 113, the resilient tongue 31 is brought into contact with the drive spring 12 arranged internally of the plunger rod 113 for generating the audible and/or tactile feedback. Stated differently, the resilient tongue 31 is arranged to, upon movement of the plunger rod 113 in the proximal direction, be brought into contact with the drive spring 12 via the through hole of the cut-out 141 of the second portion 113d for generating the audible and/or tactile feedback.

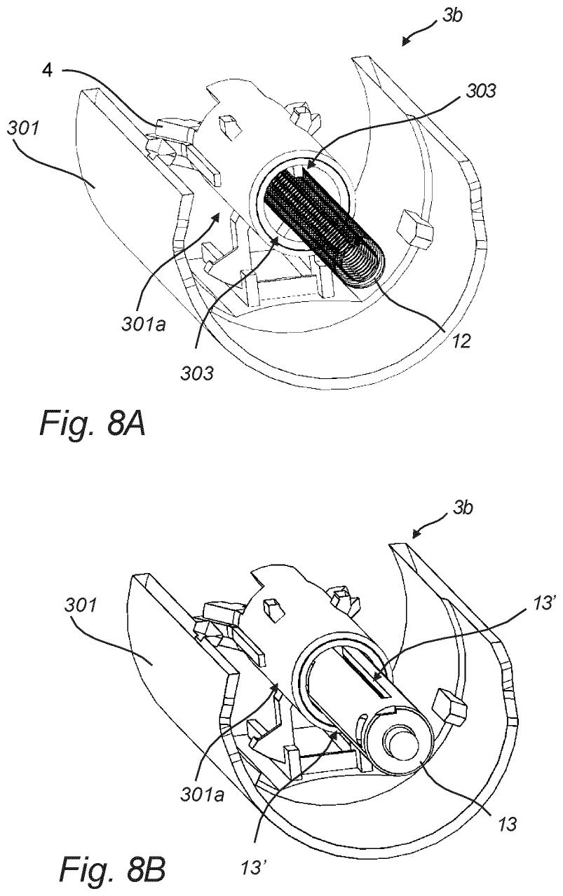

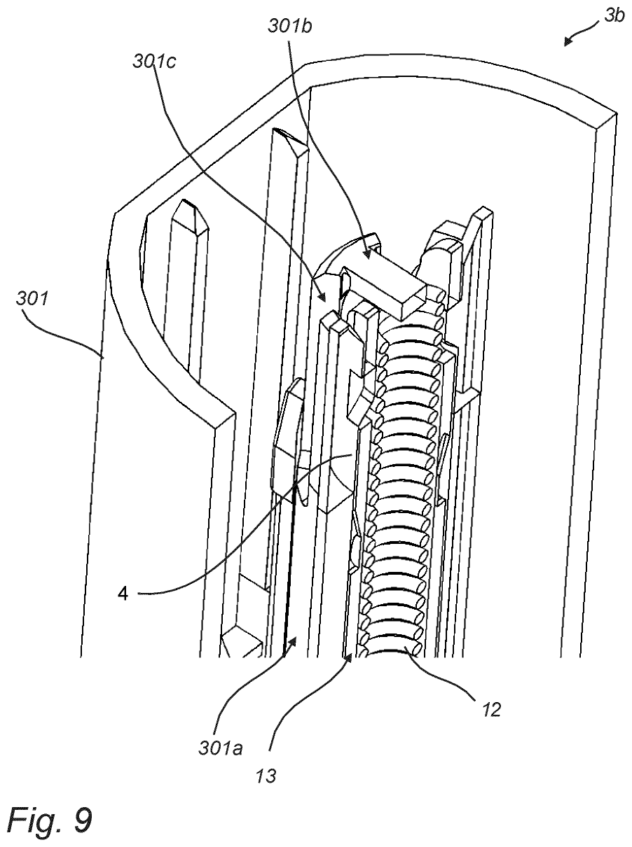

FIGS. 8A and 8B show perspective, at least partly cut-away views of a part of the medicament delivery device 1 of FIGS. 1 and 2.

In FIG. 8A, the housing structure 3, and in particular the upper housing 301, is shown together with the inner sleeve portion 301a and one of the holding arms 4. In FIG. 8A, the drive spring 12 is housed inside the inner sleeve portion 301a of the upper housing 301. The inner sleeve portion 301a comprises axially extending ribs 303 arranged axially along an inner surface of the inner sleeve portion 301a, the inner surface facing radially inwards. The axial ribs 303 extend in the longitudinal direction along the inner sleeve portion 301a, and extend in the radial direction towards the drive spring 12 to radially support the drive spring 12.

In FIG. 8B, the same view and components as in FIG. 8A is shown but with the addition of the plunger rod 13. The plunger rod 13 is arranged radially outwards of the drive spring 12, and comprises axially extending slits 13′ arranged to interact with the axial ribs 303 of the inner sleeve portion 301a shown in FIG. 8A. Thus, the axial ribs and the axially extending slits may interact with each other during the axial movement of the plunger rod 13 in the proximal direction. Hereby, the plunger rod 13 is supported within the inner sleeve portion 301a. Moreover, after a medicament delivery operation, in which the plunger rod 13 has moved proximally away from the distal end 3b of the housing structure 3, the axial ribs 303 may prevent the drive spring 12 from buckling. Two axial ribs 303 and two slits 13′ of the plunger rod 13 are shown in FIGS. 8A and 8B, but there may be more than two each, e.g. three each arranged along the circumference of the inner surface of the inner sleeve portion 301a and the plunger rod 13, respectively.

FIG. 9 show a perspective view of the housing structure 3, and in particular the upper housing 301, together with the bridge 301b. The bridge 301b is extending radially inwards from the inner sleeve portion 301a shown in FIGS. 8A and 8B. The inner sleeve portion 301a may, or may not, comprise the axial ribs 303 described with reference to FIGS. 8A and 8B. In more detail, the inner sleeve portion 301a comprises a longitudinally extending bridge arm 301c extending in the same direction as the first holding arm 4, but at a 90 degrees angle relative the first holding arm 4. The bridge 301b extends from a distal end of the bridge arm 301c radially inwards towards a longitudinal centre axis of the housing structure 3. The bridge 301b may be formed as a radially extending protrusion, or a radially extending beam. The bridge 301b extends radially inwards to a position at which it supports the drive spring 12 in the housing structure 3. The bridge 301b comprises a proximally facing surface acting as a contact surface for the drive spring 12. Thus, the drive spring 12 may be biased in the proximal direction by being compressed against the contact surface of the bridge 301b. As the bridge 301b supports the drive spring 12 within the housing structure 3, the plunger rod 13 may be arranged radially outwards of the drive spring 12.

According to some variations, the drive member could for example be motor driven or manually driven instead of having an automatic spring-loaded structure. Moreover, it is envisaged that e.g. the hinge structure according to some examples could be provided without the radially outwards extending structures, and the medicament delivery device could thus be provided without the proximal sleeve and the holding sleeve.

The inventive concept has mainly been described above with reference to a few examples. However, as is readily appreciated by a person skilled in the art, other embodiments than the ones disclosed above are equally possible within the scope of the inventive concept, as defined by the appended claims.

1-14. (canceled)

15. A feedback mechanism for a medicament delivery device, the feedback mechanism comprising:
a housing structure having a proximal end and a distal end, the housing structure comprising a holding arm arranged at the distal end of the housing structure;
a plunger rod having a proximal end and a distal end, the plunger rod comprising a holding structure arranged at the distal end of the plunger rod, the holding structure being configured to engage with the holding arm in a pre-tensioned state, wherein the plunger rod is operatively arranged to, upon activation, be released from its pre-tensioned state by disengagement of the holding structure from the holding arm of the housing structure, to move proximally inside the housing structure; and
a signal generating member attached to the housing structure, the signal generating member being arranged radially outside of the plunger rod and comprising a resilient tongue which in the pre-tensioned state of the plunger rod is arranged in contact with a first portion of the plunger rod,
wherein the plunger rod comprises a second portion arranged distally from the first portion, the second portion comprising a cut-out, and wherein the resilient tongue is operably arranged to, upon movement of the plunger rod in the proximal direction, be brought into contact with the second portion of the plunger rod to interact with the cut-out for generating an audible and/or tactile feedback.

16. The feedback mechanism according to claim 15, wherein the plunger rod is, upon activation, movable between a first end position and a second end position, and wherein the resilient tongue is arranged in pre-tensioned contact with the first portion of the plunger rod in the first end position of the plunger rod, and is arranged to interact with the cut-out for generating an audible and/or tactile feedback in the second end position of the plunger rod.

17. The feedback mechanism according to claim 15, wherein the first portion of the plunger rod comprises a cut-out which the resilient tongue is in pre-tensioned contact in the pre-tensioned state of the plunger rod.

18. The feedback mechanism according to claim 17, wherein the plunger rod comprises an external surface having an intermediate portion arranged axially between the first portion and the second portion, wherein the intermediate portion is arranged to support and flex the resilient tongue radially outwards upon movement of the plunger rod in the proximal direction.

19. The feedback mechanism according to claim 18, wherein the diameter of the plunger rod at the intermediate portion is larger than the diameter of the plunger rod at the cut out of the second portion.

20. The feedback mechanism according to claim 15, wherein the cut-out of the second portion of the plunger rod is an indentation in the plunger rod.

21. The feedback mechanism according to claim 20, wherein the resilient tongue is arranged to, upon movement of the plunger rod in the proximal direction, be brought into contact with the indentation for generating the audible and/or tactile feedback

22. The feedback mechanism according to claim 15, wherein the cut-out of the second portion of the plunger rod is a through hole in the plunger rod.

23. The feedback mechanism according to claim 15, further comprising a drive spring operably arranged to act on the plunger rod to exert a force in the proximal direction, wherein the plunger rod is arranged radially outwards of the drive spring.

24. The feedback mechanism according to claim 22, wherein the resilient tongue is arranged to, upon movement of the plunger rod in the proximal direction, be brought into contact with the drive spring via the through hole for generating the audible and/or tactile feedback.

25. The feedback mechanism according to claim 15, wherein the signal generating member comprises a sleeve surrounding the plunger rod, wherein the resilient tongue protrudes radially inwards from the sleeve.

26. The feedback mechanism according to claim 25, wherein the resilient tongue is a first resilient tongue, and the signal generating member comprises a second a resilient tongue protruding radially inwards from the sleeve opposite to the first resilient tongue.

27. The feedback mechanism according to claim 25, wherein the signal generator member comprises an attachment arm extending distally from the sleeve, wherein the attachment arm is fixedly attached to the housing structure.

28. A medicament delivery device comprising a feedback mechanism according to claim 15.
